(19) United States
(12) Patent Application Publication
(10) Pub. No.: US 2025/0262089 A1
(43) Pub. Date: Aug. 21, 2025

(54) SYSTEM AND METHOD FOR TREATING OVER-ACTIVE BLADDER

(71) Applicant: The Alfred E. Mann Foundation for Scientific Research, Valencia, CA (US)

(72) Inventors: Trent Jonathan Schramm, Castaic, CA (US); Anthony C. Ng, Santa Clarita, CA (US); Neil Hamilton Talbot, La Crescenta, CA (US)

(21) Appl. No.: 19/040,657

(22) Filed: Jan. 29, 2025

Related U.S. Application Data
(60) Provisional application No. 63/554,654, filed on Feb. 16, 2024.

(51) Int. Cl. A61F 7/02; A61F 7/00

(52) U.S. Cl. CPC A61F 7/02; A61F 2007/0042; A61F 2007/0043; A61F 2007/0044; A61F 2007/0075; A61F 2007/0093; A61F 2007/0096

(57) ABSTRACT

A method of treating a patient suffering from over-active bladder (OAB) or other urinary or bowel movement disorder includes placing at least one thermal device on an exterior portion of the patient's body proximate to the patient's saphenous nerve or the patient's tibial nerve. The thermal device includes a number of heating elements and a number of cooling elements arranged in a thermal array. The method also includes activating the heating elements and the cooling elements to stimulate the saphenous nerve and/or the tibial nerve to alleviate symptoms of the over-active bladder or the other urinary or bowel movement disorder.

### CROSS-REFERENCE TO RELATED APPLICATION(S)

The present application claims priority to and the benefit of U.S. Provisional Application No. 63/554,654, filed Feb. 16, 2024, the entire content of which is incorporated herein by reference.

### BACKGROUND

### 1. Field

The present disclosure relates to systems and methods for treating over-active bladder or other urinary or bowel movement disorder.

### 2. Description of the Related Art

A variety of different devices and methods have been developed to treat over-active bladder (OAB). For instance, related art devices and methods utilize electro-stimulation to stimulate nerves to treat OAB. However, many of these treatments are invasive and have the potential for complications or side effects.

The above information disclosed in this Background section is only for enhancement of understanding of the background of the invention and therefore it may contain information that does not constitute prior art.

### SUMMARY

The present disclosure relates to various embodiments of a method of treating a patient suffering from over-active bladder (OAB) or other urinary or bowel movement disorder. In one embodiment, the method includes placing at least one thermal device on an exterior portion of the patient's body proximate to the patient's saphenous nerve or the patient's tibial nerve. The thermal device includes a number of heating elements and a number of cooling elements arranged in a thermal array. The method also includes activating the heating elements and the cooling elements to stimulate the saphenous nerve and/or the tibial nerve to alleviate symptoms of the over-active bladder or the other urinary or bowel movement disorder.

Placing the at least one thermal device may include wrapping the thermal device around at least a portion of the patient's calf.

Placing the at least one thermal device may include wrapping the thermal device around at least a portion of the patient's knee.

Placing the at least one thermal device may include wrapping the thermal device around at least a portion of the patient's ankle.

Placing the at least one thermal device may include placing a first thermal device on the patient proximate to the saphenous nerve and placing a second thermal device on the patient proximate to the tibial nerve.

The heating elements may be hot Peltier devices, and the cooling elements may be cold Peltier devices.

The heating elements may be resistive heating elements, and the cooling elements may be cold Peltier devices.

The method may include determining a temperature of at least one heating element of the heating elements, and automatically adjusting, based on the measured temperature, a current supplied to the heating elements to achieve a desired temperature.

The method may include determining a temperature of at least one cooling element of the cooling elements, and automatically adjusting, based on the measured temperature, a current supplied to the cooling elements to achieve a desired temperature.

The method may include periodically reversing a direction of a current applied to the hot Peltier devices and the cold Peltier devices. Periodically reversing the direction of the current causes the hot Peltier devices to become cooling elements and causes the cold Peltier devices to become heating elements.

A length of time between the periodically reversing of the direction of the current to the hot Peltier devices and the cold Peltier devices may be between approximately 15 seconds and approximately 10 minutes.

This summary is provided to introduce a selection of features and concepts of embodiments of the present disclosure that are further described below in the detailed description. This summary is not intended to identify key or essential features of the claimed subject matter, nor is it intended to be used in limiting the scope of the claimed subject matter. One or more of the described features may be combined with one or more other described features to provide a workable system or method.

### BRIEF DESCRIPTION OF THE DRAWINGS

The features and advantages of embodiments of the present disclosure will be better understood by reference to the following detailed description when considered in conjunction with the drawings. The drawings are not necessarily drawn to scale.

FIG. 1 is a schematic diagram of a system for treating an over-active bladder or other urinary or bowel movement disorder according to one embodiment of the present disclosure;

FIG. 2 is a schematic block diagram of the embodiment of the system depicted in FIG. 1;

FIGS. 3A-3E depict a front view of a thermal device of the system of FIG. 2 according to various embodiments of the present disclosure;

FIG. 4 is a depiction of the principle of gate control theory; and

FIG. 5 is a flowchart illustrating tasks of a method of treating over-active bladder or other urinary or bowel movement disorder utilizing an external thermal device according to one embodiment of the present disclosure.

### DETAILED DESCRIPTION

The terminology utilized herein is for the purpose of describing particular embodiments only and is not intended to be limiting of the present disclosure. As utilized herein, the singular forms “a”, “an” and “the” are intended to include the plural forms as well, unless the context clearly indicates otherwise. As utilized herein, the term “and/or” includes any and all combinations of one or more of the associated listed items.

It will be understood that, although the terms “first”, “second”, “third”, etc., may be utilized herein to describe one or more suitable elements, components, regions, and/or sections, these elements, components, regions, and/or sections should not be limited by these terms. These terms are only utilized to distinguish one element, component, region, layer or section from another element, component, region, layer or section. Thus, a first element, component, region, or section discussed could be termed a second element, component, region, or section, without departing from the spirit and scope of the present disclosure.

It will be understood that when an element is referred to as being “on”, “connected to”, “coupled to”, or “adjacent to” another element, it can be directly on, connected to, coupled to, or adjacent to the other element, or one or more intervening element(s) may be present. In contrast, when an element is referred to as being “directly on,” “directly connected to”, “directly coupled to”, or “immediately adjacent to” another element, there are no intervening elements present.

As utilized herein, the term “substantially” and similar terms are utilized as terms of approximation and not as terms of degree, and are intended to account for the inherent deviations in measured or calculated values that would be recognized by those of ordinary skill in the art. Also, the terms “about,” “approximately,” and similar terms, when utilized herein in connection with a numerical value or a numerical range, are inclusive of the stated value and refer to within an acceptable range of deviation for the particular value as determined by one of ordinary skill in the art, considering the measurement in question and the error associated with measurement of the particular quantity (e.g., the limitations of the measurement system).

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Example embodiments of the present disclosure will now be described with reference to the accompanying drawings. In the drawings, the same or similar reference numerals refer to the same or similar elements throughout. As utilized herein, the utilize of the term “may,” when describing embodiments of the present disclosure, refers to “one or more embodiments of the present disclosure.”

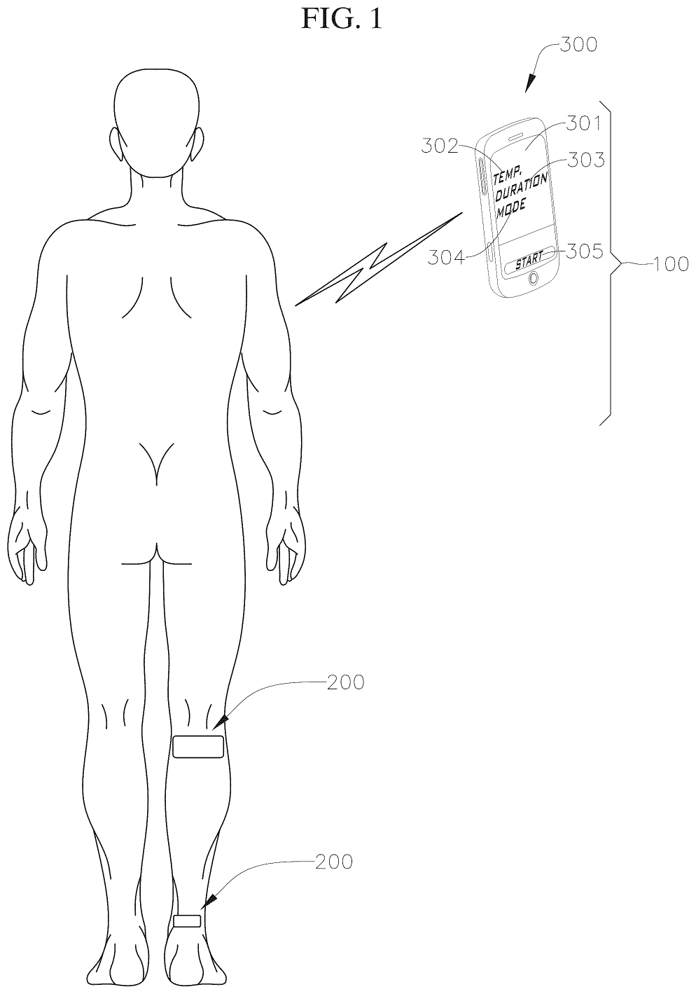

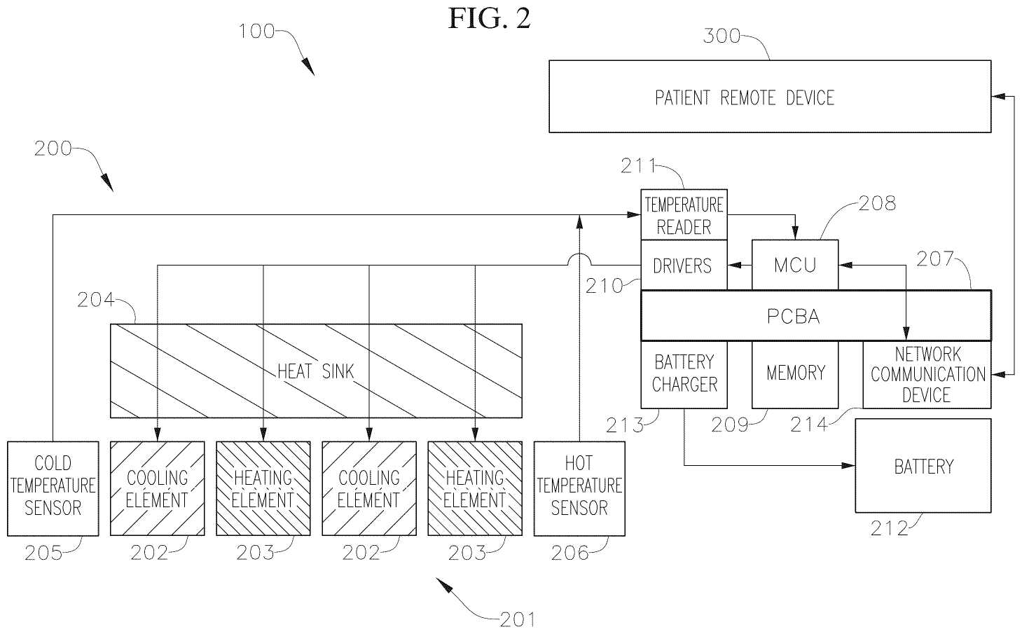

FIGS. 1-2 depict a system 100 for treating an over-active bladder (OAB) or other urinary or bowel movement disorder experienced by a patient according to one embodiment of the present disclosure. In the illustrated embodiment, the system 100 includes an external thermal device 200 and a patient remote (PR) device 300 in electronic communication with the external thermal device 200. In the illustrated embodiment, the thermal device includes a thermal array or grill 201 having a plurality of cooling elements or cooling devices 202 and a plurality of heating elements or heating devices 203. A Peltier device is a thermal control module or device that can provide either a “warming” or “cooling” effect on one surface of the device. By passing electric current through the Peltier device, it is possible to change the device surface temperature to become hotter or cooler and to maintain the desired surface temperature. When current is reversed through the Peltier device, the applicable device surface temperature can be switched from warming to cooling or from cooling to warming. In one or more embodiments, the cooling elements 202 may be Peltier devices in a cooling state and the heating elements 203 may be Peltier devices in a heating or warming state. As used herein, the term “cold Peltier” device refers to a Peltier device having an applicable surface in a cooling state the term “hot Peltier” device refers to a Peltier device having an applicable surface in a heating or warming state. Each of the cold and hot Peltier devices 202, 203 includes a core sandwiched between a pair of thermally conductive plates (i.e., a hot plate and a cool plate) and a pair of electrical leads connected to the core. The core includes an alternating arrangement of p-type and n-type semiconductor elements. When a DC current is applied via the electrical leads, a heat flux is generated at the junctions between the p-type and the n-type semiconductor elements due to the Peltier effect. When the direction of the DC current is reversed, the heat flux is reversed, which causes the hot and cold sides of the Peltier devices 202, 203 to be switched. Additionally, changing the direction of the DC current enables pulsed heating and/or cooling at any desired rate. Changing the direction of the DC current also enables switching of the hot and cold elements in the thermal array or grill 201 arrangement to avoid habituation by the user. In one or more embodiments, a length of time between subsequent tasks of reversing the direction of the current to the plurality of heating elements 203 and the plurality of cooling elements 202 is between approximately 15 seconds and approximately 10 minutes. In one or more embodiments, heating and cooling elements may be any other suitable types or kinds or heating and cooling devices. For example, in one or more embodiments, the heating elements 203 may be resistive heating elements.

In one or more embodiments, the thermal array 201 may be flexible such that the thermal array 201 is configured to conform (or substantially conform) to different anatomical portions of a patient. For instance, in one or more embodiments, the thermal array 201 may be configured to conform (or substantially conform) to a portion of the patient's leg and/or ankle to treat an over-active bladder or other urinary or bowel movement disorder, as illustrated in FIG. 1.

In the illustrated embodiment shown in FIG. 2, the external thermal device 200 also includes a heat sink 204 configured to dissipate heat from the thermal array 201. In the illustrated embodiment, the heat sink 204 is on a backside of the thermal array 201. When the external thermal device 200 is applied to the patient, the thermal array 201 faces the portion of the patient to which the thermal device 200 is applied, and the heat sink 204 faces away from the patient.

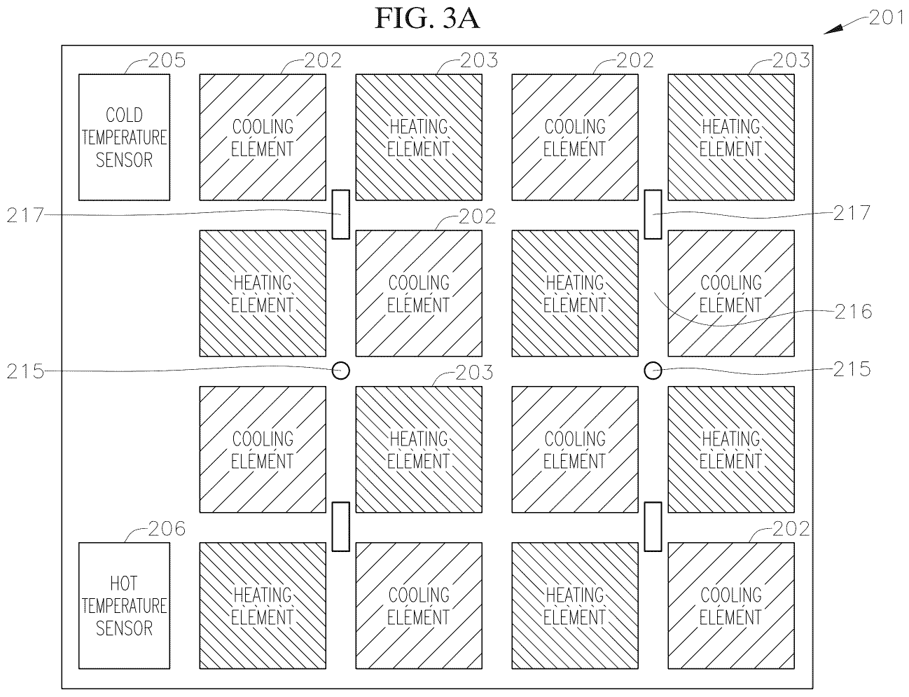

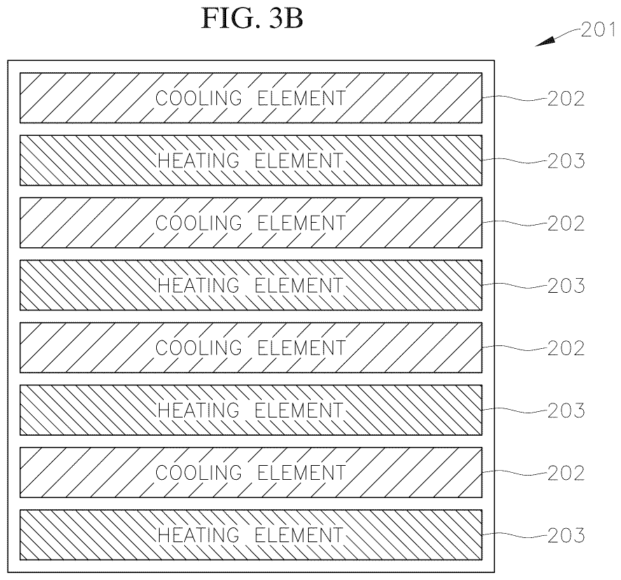

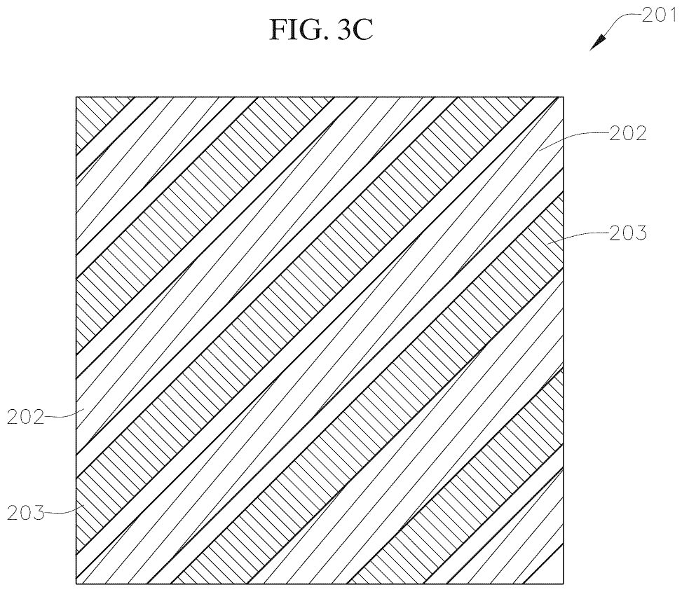

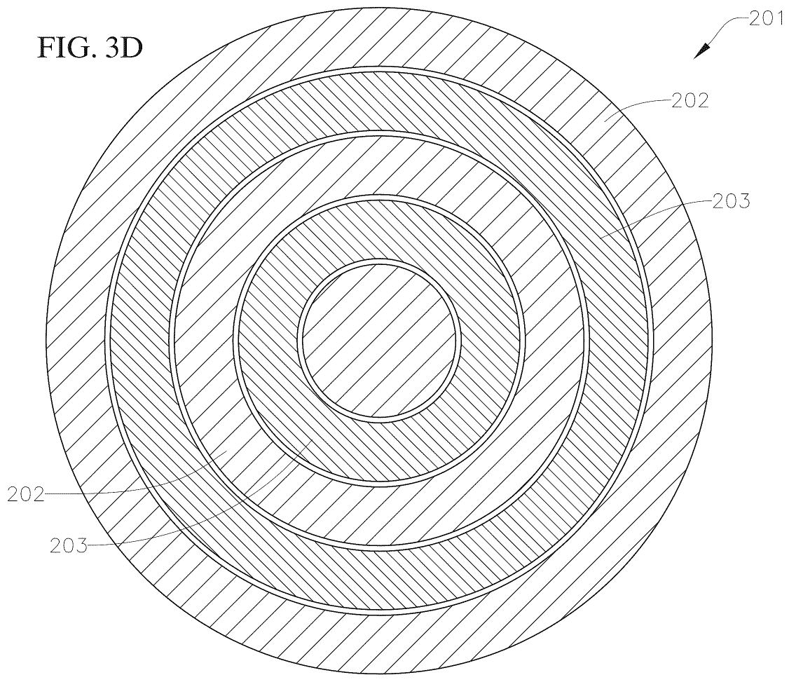

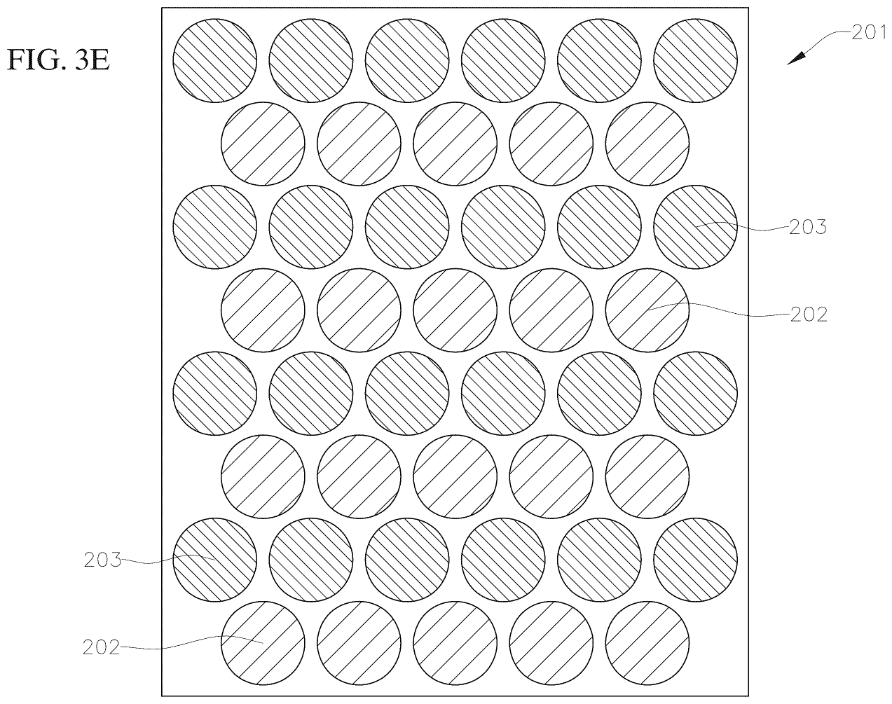

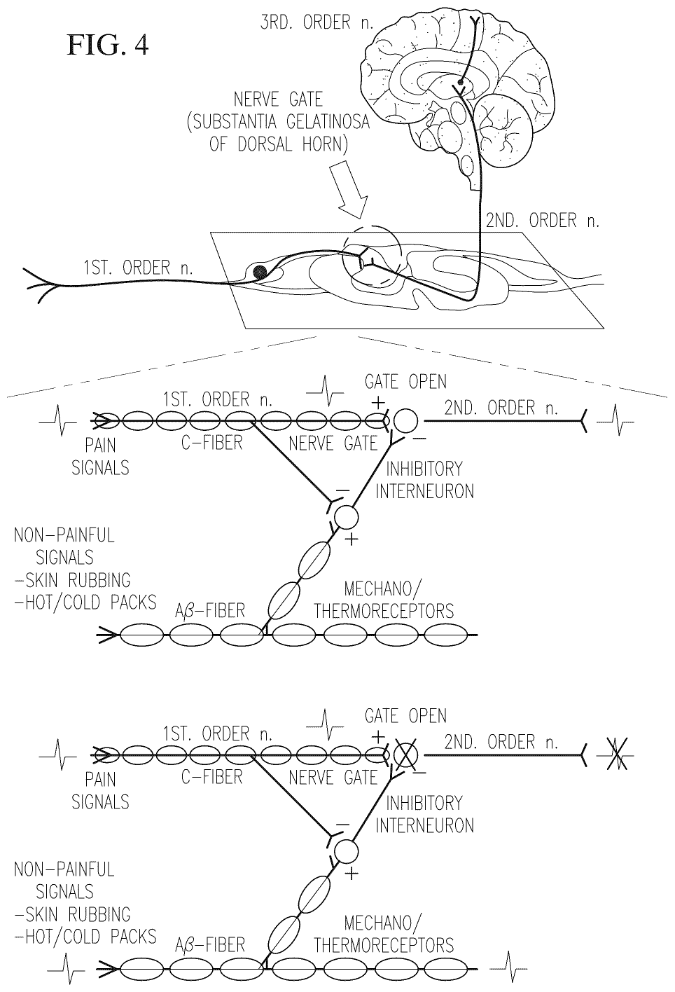

In the illustrated embodiment shown in FIG. 3A, the thermal array 201 also includes a first temperature sensor 205 connected to (or proximate to) one of the cooling elements or devices 202 (e.g., one of the cold Peltier devices), and a second temperature sensor 206 connected to (or proximate to) one of the heating elements or devices 203 (e.g., one of the hot Peltier devices).

In the illustrated embodiment shown in FIG. 2, the thermal device 200 also includes a printed circuit board (PCBA) 207, a microcontroller (MCU) 208 including at least one processor (or processor core) on the PCBA 207, a non-volatile memory device 209 (e.g., flash memory, or read-only memory (ROM), such as programmable read-only memory (PROM) or erasable programmable read-only memory (EPROM)), on the PCBA 207, one or more drivers 210 (e.g., one or more Peltier drivers) on the PCBA 207 and connected to the heating and cooling devices 202, 203, and one or more temperature readers 211 on the PCBA 207 and in electronic communication with (e.g., connected to) the first and second temperature sensors 205 and 206. The temperature sensors 205, 206 are configured to transmit or send the measured temperatures of the cooling and heating devices 202, 203, respectively, to the temperature reader 211, and the driver 210 is configured to adjust the signals (e.g., the DC voltage(s)) supplied to the cooling and heating devices 202, 203 based on the measured temperatures to achieve the desired cooling and heating temperatures provided by the cooling and heating devices 202, 203. In this manner, the thermal device 200 is configured to provide automatic closed-loop temperature control.

In the illustrated embodiment, the thermal device 200 also includes a power supply 212 (e.g., at least one battery, such as at least one secondary battery) and a battery charger 213 on the PCBA 207 and connected to the power supply 212. In one or more embodiments, the thermal device 200 may not include the power supply 212 (e.g., the battery) and thermal device 200 may instead include a power cable configured to be plugged into a wall outlet when in use. In one or more embodiments, the thermal device 200 may include both the power supply 212 (e.g., the battery) and the power cable.

The thermal device 200 also includes a network communication device (circuit) 214 (e.g., an antenna, such as a transceiver or a receiver and a transmitter) on the PCBA 207 that is configured to wirelessly communicate with the PR device 300. Wireless links may include Bluetooth™, Bluetooth Low Energy or other protocols. In one or more embodiments, the wireless communication protocol may include an authentication and encryption protocol to protect patient data. In the illustrated embodiment, the temperature reader(s) 211, the driver(s) 210, the non-volatile memory device 209, and the network communication device 214 are connected to each other over the MCU 208. In one or more embodiments, the PR device 300 may be connected to the thermal device 200 via one or more wires (e.g., a cable) and the thermal device 200 may not include the network communication device 214.

The term “processor” is utilized herein to include any combination of hardware, firmware, memory, and software, employed to process data or digital signals. Analog inputs and/or outputs to the processor may also be employed. The hardware of a processor may include, for example, a microcontroller, application specific integrated circuits (ASICs), general purpose or special purpose central processors (CPUs), digital signal processors (DSPs), graphics processors (GPUs), analog to digital converters, digital to analog converters, and programmable logic devices such as field programmable gate arrays (FPGAs). In a processor, as utilized herein, each function is performed either by hardware configured, i.e., hard-wired, to perform that function, or by more general-purpose hardware, such as a CPU, configured to execute instructions stored in a non-transitory storage medium or memory. A processor may contain two or more processors, for example, a processor may include two processors, an FPGA and a CPU, interconnected on the PCBA 207.

As shown in FIG. 1, the PR device 300 may be any suitable electronic device, such as a smartphone. Additionally, in one or more embodiments, the PR device 300 may display one or more parameters for controlling operation of the thermal device 200. In one or more embodiments, a display 301 of the PR device 300 may display a graphical user interface (GUI) including one or more buttons, sliders, or menus for controlling one or more parameters of the thermal device 200. For instance, in one or more embodiments, the GUI displayed on the PR device 300 may include a field 302 for entering the operating temperatures (or range of temperatures) of the cooling and heating devices 202, 203, a field 303 for entering the duration of operation of the thermal device 200, a field 304 for entering the operating mode of the thermal device 200, and a button 305 for activating or deactivating the thermal device 200.

FIGS. 3A-3E depict different configurations and arrangements of the cooling and heating devices 202, 203 (e.g., the hot and cold Peltier devices which may be reversed from cold-to-hot and hot-to-cold) according to various embodiments of the present disclosure. In the embodiment illustrated in FIG. 3A, the cooling and heating devices 202, 203 are square-shaped and are alternately arranged in a grid pattern including a series of rows and a series of columns (i.e., a checkerboard pattern). In the embodiment illustrated in FIG. 3B, the cooling and heating devices 202, 203 are rectangle-shaped and are alternately arranged in a series of alternating parallel (or substantially parallel) stripes. Although in the embodiment illustrated in FIG. 3B the cooling and heating devices 202, 203 are arranged vertically, in one or more embodiments the cooling and heating devices 202, 203 may be arranged in any other suitable orientation, such as horizontally. In the embodiment illustrated in FIG. 3C, the cooling and heating devices 202, 203 are trapezoid-shaped and are arranged in a series of alternating slanted (e.g., diagonal) stripes. In the embodiment illustrated in FIG. 3D, the cooling and heating devices 202, 203 are ring-shaped (i.e., annular) and are arranged in a series of concentric (or substantially concentric) rings. In the embodiment illustrated in FIG. 3E, the cooling and heating devices 202, 203 are dot-shaped (e.g., circular) and are arranged in a series of rows. Each row includes a plurality of heating elements 203 or a plurality of cooling elements 202, and the rows that include the heating elements 203 are alternately arranged with the rows that include the cooling elements 202. Although in the illustrated embodiment the dot-shaped cooling and heating elements 202, 203 are arranged in a series of horizontal rows, in one or more embodiments the dot-shaped cooling and heating elements 202, 203 may be arranged in any other suitable configuration, such as a checkerboard pattern. In one or more embodiments, the thermal device 200 may have a width in a range from approximately 0.5 inches to approximately 24 inches, and a height in a range from approximately 0.5 inches to approximately 24 inches (e.g., in one embodiment, the thermal device 200 may have a width of approximately 8 inches and a height of approximately 6 inches). In one or more embodiments, the thermal device 200 may be as small as approximately 0.5 inches×approximately 0.5 inches, and the thermal device 200 may be as large as approximately 24 inches×approximately 24 inches. Additionally, in one or more embodiments, the thermal device 200 may have a rectangular shape, a square shape, a circular shape, or any other shape suitable for the anatomical area on which the thermal device 200 is intended to be applied. Furthermore, although in one or more embodiments, the thermal device 200 includes a plurality of cooling elements 202 and a plurality of heating elements 203, in one or more embodiments the thermal device 200 may include a single thermal element that can be powered to alternately heat and cool over time, e.g., a Peltier device. While a single thermal element cannot induce the Thermal Grill Effect, the thermal device 200 would still be configured to provide the other benefits of heating and cooling in a controlled, timed, and alternating manner. In one or more embodiments, the thermal device 200 may include a single cooling element 202 and a single heating element 203. It should be understood that, in any of these embodiments, the heat and cooling may be interchanged, or pulsed from hot to cold and from cold to hot.

As shown in FIG. 3A, in one or more embodiments, the thermal device 200 may include one or more light-emitting elements 215 (e.g., one or more light-emitting diodes (LEDs) or laser diodes) in one or more of the gaps 216 between adjacent cooling and heating elements 202, 203 (e.g., a gap 216 between one cooling element 202 and an adjacent heating element 203). In one or more embodiments, the light-emitting elements 215 may be configured to emit light having a wavelength in the range from approximately 500 nm to approximately 1,200 nm. The light emitted from the light-emitting elements 215 is configured to promote healing. In one or more embodiments, the cooling and heating elements 202, 203 may be substantially transparent to permit the transmission of light from the light-emitting elements through the majority of the area of the thermal device 200. In an embodiment in which the cooling and heating elements 202, 203 are Peltier heating/cooling elements, the outer sheets of material, typically opaque alumina, could be made of a clear material, such as single-crystal alumina (sapphire) or a relatively thermally conductive glass. Additionally, in one or more embodiments, the P-N junctions inside the Peltier devices may not be transparent, but the P-N junctions may be reduced in number and spaced further apart to allow light to pass around them.

In one or more embodiments, the thermal device 200 may include one or more vibration elements 217 (e.g., one or more ultrasound elements, low frequency vibration elements, electrical stimulation elements, or electrical muscle stimulation (EMS) (i.e., electromyostimulation) elements). In one or more embodiments, the vibration elements 217 may be configured to generate vibrations in a range from approximately 1 Hz to approximately 20,000 Hz. In one or more embodiments, the vibration elements 217 may be configured to generate vibrations in the ultrasonic range above approximately 20,000 Hz. In one or more embodiments, the vibration elements 217 may be configured to generate vibrations in a range from approximately 5 Hz to approximately 1,000 Hz. The one or more vibration elements 217 are configured to provide additional nerve stimulation and/or an analgesic effect and promote blood circulation in the patient to promote healing. EMS can enhance local circulation and support healing by using specific frequency and parameter settings. For circulation improvement, the EMS may operate within a frequency range of approximately 10 Hz to approximately 30 Hz, which encourages gentle muscle contractions that promote blood flow. Pulse width ranges from approximately 200 microseconds (μs) to approximately 400 μs are configured to maintain comfort and effective stimulation without muscle fatigue (or substantially without muscle fatigue). A short duty cycle, such as approximately 2 seconds on and approximately 2 seconds off, mimics natural muscle rhythms, enhancing circulation without causing strain. Regular sessions lasting approximately 15 minutes to approximately 20 minutes at moderate intensity can improve tissue oxygenation, expedite healing, and reduce recovery time in targeted areas. Transcutaneous electrical nerve stimulation (TENS) and microcurrent electrical neuromuscular stimulation (MENS) can provide similar benefits to EMS and can also be used in conjunction with the thermal grill effect to reduce pain and promote healing.

In operation, the PR device 300 shown in FIG. 1 is configured to send (e.g., transmit) a signal to the thermal device 200 to operate the cooling and heating elements 202, 203. The signal may be transmitted, for example, in response to a user entering a command (e.g., pushing button 305) on the GUI displayed on the PR device 300. In one or more embodiments, the signal from the PR device 300 may be received by the network communication device 214 (in FIG. 2). In one or more embodiments, the signal from the PR device 300 may be received by a wire or cable connected to the thermal device 200. In one or more embodiments, the non-volatile memory device 209 of the thermal device 200 includes instructions which, when executed by the MCU 208 in response to receipt of the signal from the PR device 300, cause the driver(s) 210 to deliver DC current(s) from the power supply 212 to the cooling and heating elements 202, 203. The DC current causes the heating elements 203 to generate heat at the side adjacent to the skin and causes the cooling elements 202 to remove heat from the side adjacent to the skin.

Together, the heat generated by the heating elements 203 and the thermal dissipation caused by the cooling elements 202 causes a thermal grill effect (i.e., a thermal grill illusion) when the thermal device 200 is applied to a patient. The thermal grill illusion refers to a sensory illusion in which the interlacing of hot and cold elements causes a slightly painful sensation in a healthy subject without a noxious stimulus (i.e., the thermal grill illusion activates a region of the brain associated with noxious thermal stimuli to generate a pain sensation). In one or more embodiments, the thermal grill illusion can be achieved with very mild hot and cold temperatures, for example, in the range of approximately 5° C. to approximately 20° C. for the cooling elements 202 and in the range of approximately 38° C. to approximately 50° C. for the heating elements 203. In one embodiment, the thermal grill illusion can be achieved with temperatures in the range of approximately 15° C. to approximately 20° C. for the cooling elements 202 and approximately 38° C. to approximately 44° C. for the heating elements 203. Throughout the present disclosure, the heating elements 203 are often described as hot, but in practice temperatures generated by the heating elements 203 would typically not exceed approximately 44° C.

The thermal grill effect or illusion generated by the thermal device 200 may be utilized to treat or alleviate an over-active bladder or other urinary or bowel movement disorder. For instance, in one or more embodiments, this sensory illusion may stimulate the saphenous nerve and/or the tibial nerve depending on the location of the thermal device on the patient's body, and this stimulation of the saphenous nerve and/or the tibial nerve may alleviate the symptoms of an over-active bladder or other urinary or bowel movement disorder.

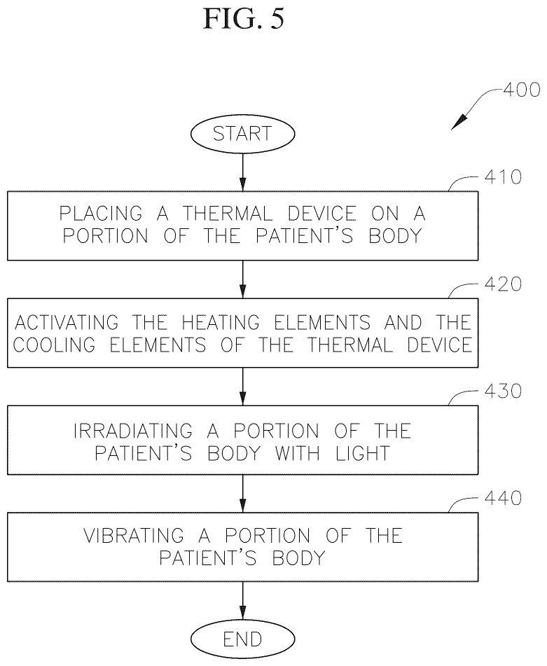

FIG. 5 is a flowchart depicting tasks of a method 400 of treating a patient suffering from an over-active bladder (OAB) or other urinary or bowel movement disorder. In the illustrated embodiment, the method 400 includes a task 410 of placing a thermal device (e.g., an embodiment of the thermal device depicted in FIGS. 1-3E) on an exterior portion of the patient's body proximate to the patient's saphenous or tibial nerves or both nerves. The saphenous nerve runs down the back of the leg. The tibial nerve originates from the L4, L5, S1, S2, and S3 spinal nerve roots and is the larger terminal branch of the sciatic nerve. The tibial nerve runs downward and medially along the back of the leg to the posteromedial side of the ankle. In one or more embodiments, the task 410 of applying the thermal device varies depending on whether the saphenous nerve and/or the tibial nerve will be stimulated. In one or more embodiments, the task 410 of applying the thermal device includes placing the thermal device on a posterior portion of one of the patient's legs (e.g., placing the thermal device on a portion of the patient's calf or behind the knee). In one or more embodiments, the task 410 of applying the thermal device includes placing the thermal device on a portion of one of the patient's ankles (e.g., placing the thermal device on the medial malleolus of one of the patient's ankles). In one or more embodiments, the task 410 may include partially or completely wrapping the thermal device around one of the patient's leg and/or one of the patient's ankle. Additionally, in one or more embodiments, the task 410 may include applying or placing a first thermal device on a posterior portion of one of the patient's legs and applying or placing a second thermal device on a portion of one of the patient's ankles.

In the illustrated embodiment, the method 400 also includes a task 420 of activating the thermal device. In one or more embodiments, the task 420 of activating the thermal device includes simultaneously (or substantially simultaneously) activating the heating elements and the cooling elements of the thermal device (e.g., activating the hot and cold Peltier devices). In one or more embodiments, the task 420 of activating the thermal device may include determining, with a temperature sensor, a temperature of at least heating element of the plurality of heating elements, and automatically adjusting, based on the measured temperature, a current supplied to the plurality of heating elements to achieve a desired temperature. Similarly, in one or more embodiments, the task 420 of activating the thermal device may include determining, with a temperature sensor, a temperature of at least cooling element of the plurality of cooling elements, and automatically adjusting, based on the measured temperature, a current supplied to the plurality of cooling elements to achieve a desired temperature. As described above, simultaneously generating heat from the heating elements and cooling from the cooling elements causes the patient to experience the thermal grill illusion (TGI), which is a sensory illusion in which the patient experiences a mild pain sensation in the absence of a noxious stimulus. This TGI is configured to stimulate the saphenous nerve and/or the tibial nerve depending on where the thermal device was placed in task 410. The stimulation of the patient's saphenous nerve and/or the patient's tibial nerve is configured to alleviate or at least mitigate the patient's symptoms of an over-active bladder or other urinary or bowel movement disorder.

The system, any other relevant devices or components, and the method according to embodiments of the present disclosure described herein may be implemented utilizing any suitable hardware, firmware (e.g., an application-specific integrated circuit), software, or a combination of software, firmware, and hardware. For example, the one or more suitable components of the system may be formed on one integrated circuit (IC) chip or on separate IC chips. Further, the one or more suitable components of the system may be implemented on a flexible printed circuit film, a tape carrier package (TCP), a printed circuit board (PCB), or formed on one substrate. Further, the one or more suitable components of the system may be a process or thread, running on one or more processors, in one or more computing devices, executing computer program instructions and interacting with other system components for performing the one or more suitable functionalities described herein. The computer program instructions are stored in a memory which may be implemented in a computing device utilizing a standard memory device, such as, for example, a random access memory (RAM). The computer program instructions may also be stored in other non-transitory computer readable media such as, for example, a flash drive, and/or the like. Also, a person of skill in the art should recognize that the functionality of one or more suitable computing devices may be combined or integrated into a single computing device, or the functionality of a particular computing device may be distributed across one or more other computing devices without departing from the scope of the example embodiments of the present disclosure.

Although some embodiments of the present disclosure have been disclosed herein, the present disclosure is not limited thereto, and the scope of the present disclosure is defined by the appended claims and equivalents thereof.

What is claimed is:

1. A method of treating a patient suffering from an over-active bladder or other urinary or bowel movement disorder, the method comprising:
placing at least one thermal device on an exterior portion of the patient's body proximate to the patient's saphenous nerve and/or the patient's tibial nerve, the at least one thermal device comprising a plurality of heating elements and a plurality of cooling elements arranged in a thermal array; and
activating the plurality of heating elements and the plurality of cooling elements to stimulate the saphenous nerve and/or the tibial nerve to alleviate symptoms of the over-active bladder or the other urinary or bowel movement disorder.

2. The method of claim 1, wherein the placing the at least one thermal device comprises wrapping the thermal device around at least a portion of the patient's calf.

3. The method of claim 1, wherein the placing the at least one thermal device comprises wrapping the thermal device around at least a portion of the patient's knee.

4. The method of claim 1, wherein the placing the at least one thermal device comprises wrapping the thermal device around at least a portion of the patient's ankle.

5. The method of claim 1, wherein the placing the at least one thermal device comprises placing a first thermal device on the patient proximate to the saphenous nerve and placing a second thermal device on the patient proximate to the tibial nerve.

6. The method of claim 1, wherein the plurality of heating elements is a plurality of hot Peltier devices, and wherein the plurality of cooling elements is a plurality of cold Peltier devices.

7. The method of claim 1, wherein the plurality of heating elements is a plurality of resistive heating elements, and wherein the plurality of cooling elements is a plurality of cold Peltier devices.

8. The method of claim 1, further comprising:
determining a temperature of at least one heating element of the plurality of heating elements; and
automatically adjusting, based on the measured temperature, a current supplied to the plurality of heating elements to achieve a desired temperature.

9. The method of claim 1, further comprising:
determining a temperature of at least one cooling element of the plurality of cooling elements; and
automatically adjusting, based on the measured temperature, a current supplied to the plurality of cooling elements to achieve a desired temperature.

10. The method of claim 6, further comprising periodically reversing a direction of a current applied to the plurality of hot Peltier devices and the plurality of cold Peltier devices, wherein the periodically reversing the direction of the current causes the plurality of hot Peltier devices to become cooling elements and causes the plurality of cold Peltier devices to become heating elements.

11. The method of claim 10, wherein a length of time between the periodically reversing of the direction of the current to the plurality of hot Peltier devices and the plurality of cold Peltier devices is between approximately 15 seconds and approximately 10 minutes.
